(19) United States
(12) Patent Application Publication
(10) Pub. No.: US 2020/0227165 A1
(43) Pub. Date: Jul. 16, 2020

(54) COGNITIVE PROCESSING SYSTEM

(71) Applicant: ForeSee Medical, Inc., San Diego, CA (US)

(72) Inventors: Seth Flam, San Diego, CA (US); Rumpa Giri, Solana Beach, CA (US); Arnold Mentze, San Marcos, CA (US)

(21) Appl. No.: 16/245,192

(22) Filed: Jan. 10, 2019

(51) Int. Cl. G16H 50/20; G16H 50/30; G16H 10/60

(52) U.S. Cl. CPC G16H 50/20; G16H 10/60; G16H 50/30

(57) ABSTRACT

A system and method include discovering diseases associated with a patient. The method includes receiving, by one or more processors and from a client device, a request for a health assessment of a patient; aggregating, by one or more processors and from one or more sources, patient data associated with the patient; scanning, by the one or more processors, the patient data to generate patient disease data for the patient data that includes a list of characteristics identifying a disease that has not been coded in the patient data, the scanning based on a database of parser rules corresponding to interoperability standards and inspection rules corresponding to disease symptoms; and transmitting, by the one or more processors, the patient disease data to the client device to cause the patient disease data to be presented via the client device.

### BACKGROUND

The following description is provided to assist the understanding of the reader. None of the information provided or references cited is admitted to be prior art.

Traditional software systems suffer from several technical challenges that limit the capabilities of the system. For example, software is limited to the information given it, which may or may not be in a structured and common format. Beyond limitations of the data that software uses, analysis performed by the software often lacks the flexibility and modifiability to make determinations using imperfect, incomplete, and conflicting data inputs. Despite recent attempts in artificial intelligence (AI) and machine learning (ML), modern software systems continue to struggle in its ability to demonstrate cognition, which is the process of acquiring knowledge and understanding.

Traditional software systems in the medical industry, for example, often struggle from such technical challenges. In the medical industry, physicians must synthesize dozens of documents. Each document can be multiple pages and they change frequently. Furthermore, the documents change each time a patient visits a provider and a new treatment or lab test is ordered. Further complicating matters, one patient relates to around seven or more doctors per year, however each doctor has their own version of that patient's health record. Some of those records are exported from electronic health records (EHRs) in a special format called a Clinical Document Architecture (CCDA) may span 50 pages or more. Traditionally, it is the responsibility of each provider to consolidate those records into a single “medical chart,” which is a complex task. Weeding out duplicates that may have been sent back and forth between providers and discovering new findings is a time-consuming effort. Traditionally, EHR software does not do the job with regard to consolidating the patient record into an easy to read, accurate format. Without this capability, medical providers will inevitably “miss” an important finding thereby exposing themselves to increased malpractice risk. There is a need for a cognitive processing system that overcomes these challenges to help in discovery of diseases for patients.

### SUMMARY

In accordance with some aspects of the present disclosure, a computer-based method for discovering diseases associated with a patient is disclosed. The method includes receiving, by one or more processors and from a client device, a request for a health assessment of a patient; aggregating, by one or more processors and from one or more sources, patient data associated with the patient; scanning, by the one or more processors, the patient data to generate patient disease data for the patient data that includes a list of characteristics identifying a disease that has not been coded in the patient data, the scanning based on a database of parser rules corresponding to interoperability standards and inspection rules corresponding to disease symptoms; and transmitting, by the one or more processors, the patient disease data to the client device to cause the patient disease data to be presented via the client device.

In accordance with some other aspects of the present disclosure, a system is disclosed. The system includes one or more processors performing a process including receiving, from a client device, a request for a health assessment of a patient; aggregating, from one or more sources, patient data associated with the patient; scanning the patient data to generate patient disease data for the patient data that includes a list of characteristics identifying a disease that has not been coded in the patient data, the scanning based on a database of parser rules corresponding to interoperability standards and inspection rules corresponding to disease symptoms; and transmitting the patient disease data to the client device to cause the patient disease data to be presented via the client device.

In accordance with yet other aspects of the present disclosure, a non-transitory computer readable media with computer-executable instructions embodied thereon is disclosed. The instructions when executed by a processor of an authentication system cause the authentication system to perform a process. The process includes receiving, from a client device, a request for a health assessment of a patient; aggregating, from one or more sources, patient data associated with the patient; scanning the patient data to generate patient disease data for the patient data that includes a list of characteristics identifying a disease that has not been coded in the patient data, the scanning based on a database of parser rules corresponding to interoperability standards and inspection rules corresponding to disease symptoms; and transmitting the patient disease data to the client device to cause the patient disease data to be presented via the client device.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 is an example block diagram of a cognitive processing system, in accordance with some embodiments of the present disclosure.

FIG. 2 is an example flow diagram of a cognitive process system of the system of FIG. 1, in accordance with some embodiments of the present disclosure.

FIG. 3 is another example flow diagram of fulfilling requests regarding identifying diseases associated with a patient, in accordance with some embodiments of the present disclosure.

FIG. 4 is an example block diagram of a disease discovery system, in accordance with some embodiments of the present disclosure.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

The present disclosure is directed to a cognitive processing software system designed to overcome technical challenges of acquiring meaningful information and making correct assessments from that information. The disclosure describes a system designed to properly store or access only relevant data related to a subject of interest and applying standard and custom rules against the data set. The subject of interest in the medical field can be a patient.

The present disclosure is further directed to a cognitive processing software system designed to identify care gaps, outliers, and trends and then communicate via a robust alert system that provides easy to understand analysis of the data. To achieve the technical solutions, the software system includes a rule engine that satisfies complex guidelines involving the management of patient populations but simple enough that a physician can use it without the support of an engineer. A simplified rules engine interface enables physicians to create personal and precise ways to manage their patient populations.

The cognitive programming software system also enables decision support for the discovery of diseases (referred to as the patient's disease list) that have not been coded as part of the patient record. The software also advantageously applies Medicare HCC (Hierarchical Condition Category) logic to the patient disease list and provides compliance with Clinical Quality Measures (CQMs). The software system compares the performance of providers with regard to Clinical Quality and Resource Use and compares the Disease Burden of Patients to improve patient engagement.

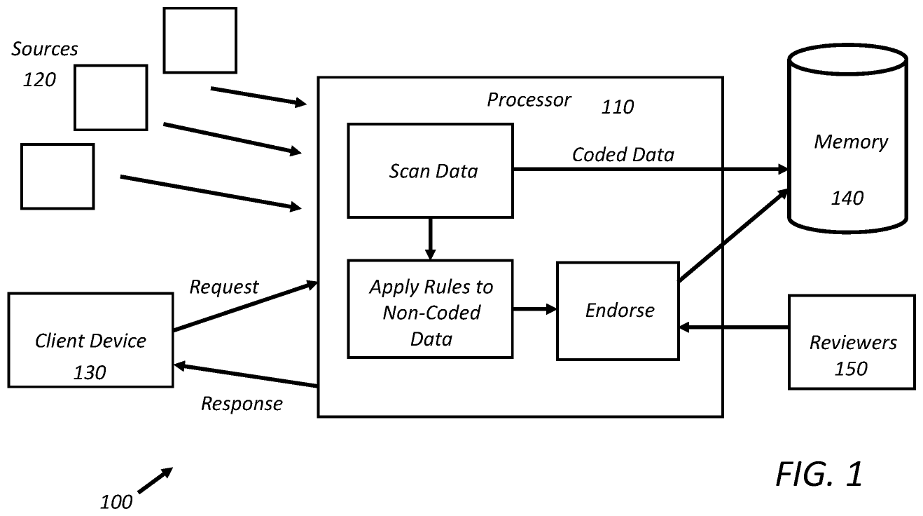

FIG. 1 illustrates a system 100 including a processor 110 that receives data from sources 120 and a request from client device 130. The processor 110 receives data from sources 120 including patient information, encounter notes, and other medical related information. Sources 120 may communicate the information electronically over a network or the processor 110 may receive the information from scanned copies of paper records. Sources 120 can include electronic medical records (EMRs), pharmacies, payers, labs, radiology, medical devices, patient portals, fitness devices, health information networks, and other sources of medical information. The request from client device 130 is an electronic request for patient information and possible diagnosis information received via a network.

The processor 110, executing a cognitive processing software, creates and manages a longitudinal patient record. According to an exemplary embodiment, the software can create such a record by aggregating patient data from multiple sources 120 including both structured and non-structured data to extract terms that help define the patient's medical data. Unstructured data can be compiled using natural language processing algorithms.

Structured data read by the software can include Vital Signs, Lab Data—LOINC Codes, Medications—RxNorm Codes, Problem List—SNOMED Codes, Smoking Status—SNOMED Codes, Medication Allergies—SNOMED/RxNorm Codes, Procedures—CPT and HCPCS Codes, Immunizations—CVX Codes, Unique Device Identifier(s)—UDI Code, Race and Ethnicity—CDC Race and Ethnicity Code Set, and Preferred Language. The structured data is stored in a memory 140 that is associated with the processor 110.

Unstructured data read by the software system and converted to structured data using natural language processing can include extracted terms from PDF or Text medical notation documents, applied dictionary of abbreviations and synonyms, noted terms that are negated or hypothetical, noted terms that are historic or family history, convert prose (keyword phrases) to structured (codified) data, group findings by the section they were discovered in the medical note, correlated results with tests and studies (e.g., a CT scan showed a tumor—the study is the “CT scan” and the result is “a tumor”), and application of post-extraction processing rules.

The software system creates sets of data (referred to as value sets) that may be referred to in a clinical rules engine. The value sets may be a variety of types such as laboratory, medication, procedure, problem list (structured data), and keywords (unstructured data). Structured data value sets can be formed by grouping sets of codes from one of the structured data sets, grouping codes based on their relevancy to one or more diseases, including the code description and numeric code, and naming a value set with a unique value set ID and value set name. Unstructured data sets can be formed by grouping sets of words or phrases (keywords) extracted via the NLP engine, grouping keywords based on their relevancy to one or more diseases, and naming a code set and label with a unique numeric code.

After the patient data from sources 120 is aggregated, the software system uses processor 110 to normalize and harmonize the data such that the data conforms to a common format. The software system uses the data to create a longitudinal patient record, which is a record of data elements and a corresponding date, and stored in memory 140. The longitudinal patient record data can then be used in a medical disease discovery rules engine.

According to an exemplary embodiment, the cognitive processing software executed by processor 110 converts expert medical knowledge about the characteristics of diseases to a set of rules. The software system then rates each rule based on its uniqueness or relevance to a specific disease. In at least one embodiment, the software system creates rules using a rubric containing definitions of specific diseases, applying a value set, setting a threshold value, and rating the relative strength of each rule to express how relevant and specific the rule is to disease. Rules that are not exclusive to a specific disease may be endorsed based on a rating system by other rules that are not exclusive to a specific disease to improve relevance of a set of rules to a specific disease. Endorsements may come from third party reviewers 150 over a network interface such as a web portal. The software system preferably rates diseases based on whether they persist for the lifetime of the patient or whether the disease may be cured or event driven (e.g., acute heart attack).

Once the rules and rating are done, the software system applies the patient's longitudinal patient record to the disease discovery rules engine to create a list of diseases for each patient record. Each disease or Clinical Quality Measure (CQM) may have many associated rules. Each rule preferably has an associated value set and each value set may have many codes. When longitudinal patient record data matches a rule, then the rule is “activated.” An activated rule may be subject to a time concurrency constraint. Rules that are activated and pass time concurrency constraints are then subject to post-processing logic.

Advantageously, the cognitive processing software system includes a medical post-processing logic rules engine, based on certain coding rules, to exclude diseases that may meet the definition of disease based on expert medical knowledge, but do not meet the coding rule criteria. In some embodiments, diseases discovered may “expire” because they are not likely to persist past the measurement period. The measurement period for Clinical Quality Measures (CQMs) is defined by the measure author. The measurement period for Medicare HCC (Hierarchical Condition Category) disease discovery is the calendar year. A “persistence” rating is associated for each disease or measure that the rules engine supports. Activated rules regarding a disease that may not persist past the measurement period are discounted or nullified.

Advantageously, the cognitive processing software system uses keyword analysis of unstructured data to try to discover the International Classification of Diseases (ICD) code that is the best ICD code to define each of the patient's diseases.

Advantageously, the cognitive processing software system uses keyword analysis of data to find numerator value data that correspond to clinical quality measures. The software system also applies risk adjustment and Clinical Quality Measure (CQM) rule logic (both defined by third parties like CMS and HEDIS) to the patient disease list and numerator value data. The risk adjustment determines a risk score and for CQMs calculate numerator/denominator data (ratio of patients that meet the CQM (numerator) divided by the patients that were eligible to meet the numerator (denominator).

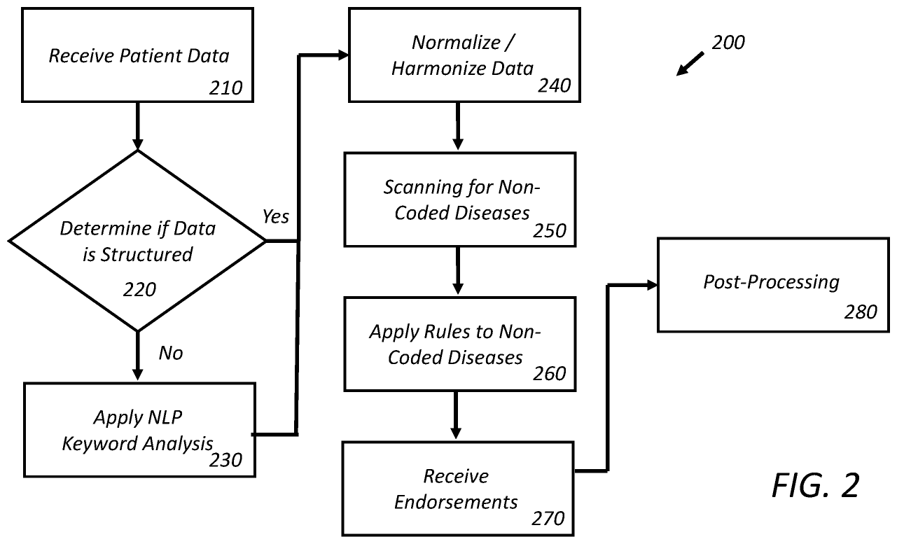

FIG. 2 illustrates a method 200 for exemplary operations performed in a process of discovering diseases associated with a patient. Additional, fewer, or different operations may be performed depending on the embodiment. In an operation 210, one or more processors receive patient data from sources including, for example, electronic medical records (EMRs), pharmacies, payers, labs, radiology, medical devices, patient portals, fitness devices, health information networks, and other sources of medical information. The one or more processors determine in operation 220 if data from the sources are structured or not. Structured data refers to data conforming to a known format and having category codes (e.g, ICD codes) that indicate to what the data refers. Multiple formats may be useable by the system. If the data is not structured, in operation 230, the one or more processors apply natural language algorithms and keyword analysis to the non-structured data.

In an operation 240, the one or more processors normalize and harmonize the data. The normalization is preferably based on parser rules and takes a first set of data extracted from the patient data and a second set of data extracted from the patient data to generate normalized data. Additional normalization techniques may be utilized. The one or more processors also use inspection rules to identify a disease from the normalized data.

The one or more processors in operation 250 scan the normalized patient data to generate patient disease data for the patient data including a list of characteristics identifying a disease that has not been coded in the patient data. The scanning follows a database of parser rules corresponding to interoperability standards and inspection rules corresponding to disease symptoms.

The one or more processors in operation 260 apply rules to the non-coded diseases to be able to sort and categorize the non-coded diseases as if they had been coded. These rules include information about assignments of diseases to standard codes. In operation 270, the one or more processors receive endorsements. Rules that are not exclusive to a specific disease may be endorsed based on a rating system by other rules that are not exclusive to a specific disease to improve relevance of a set of rules to a specific disease. Endorsements may come from third party reviewers. The system preferably rates diseases based on whether they persist for the lifetime of the patient or whether the disease may be cured or event driven (e.g., acute heart attack). The one or more processors can also in operation 280 engage a post-processing logic rules engine, based on certain coding rules, to exclude diseases that may meet the definition of disease based on expert medical knowledge, but do not meet the coding rule criteria.

Advantageously, method 200 enables the automatic classification of disease data from a patient using identified characteristics not coded in the patient data. The classification utilizes parser rules corresponding to interoperability standards and inspection rules corresponding to disease symptoms.

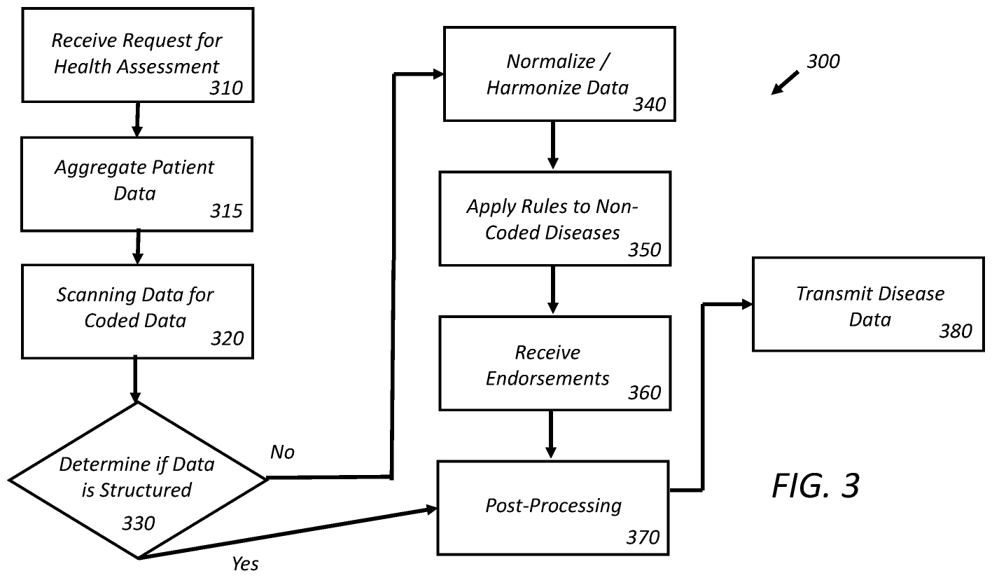

FIG. 3 illustrates a method 300 for exemplary operations performed in a process of fulfilling requests regarding identifying diseases associated with a patient. Additional, fewer, or different operations may be performed depending on the embodiment. In an operation 310, one or more processors receive a request for a health assessment for a particular patient. This request may be communicated over a closed network or using encrypted secure connections over the Internet. The request generally comes from a health care provider.

The one or more processors aggregate data associated with the patient from a plurality of sources in an operation 315. The aggregated data forms a longitudinal patient record including patient information from multiple sources such as electronic medical records (EMRs), pharmacies, payers, labs, radiology, medical devices, patient portals, fitness devices, health information networks, and other sources of medical information. In an operation 320, the one or more processors scan the data from these sources for data that is coded with standard medical codes.

The one or more processors determine if data in the patient record is structured in operation 330 and if not, the unstructured data is normalized and harmonized in operation 340. The normalization of the data may include the use of natural language algorithms. Then, in operation 350, the one or more processors apply rules to non-coded diseases to assign codes and categories to those diseases in the record that do not have such codes. In some embodiments, the one or more processors receive endorsements in operation 360. Rules that are not exclusive to a specific disease may be endorsed based on a rating system by other rules that are not exclusive to a specific disease to improve relevance of a set of rules to a specific disease. Endorsements may come from third party reviewers. The system preferably rates diseases based on whether they persist for the lifetime of the patient or whether the disease may be cured or event driven (e.g., acute heart attack). The one or more processors can also in operation 370 engage a post-processing logic rules engine, based on certain coding rules, to exclude diseases that may meet the definition of disease based on expert medical knowledge, but do not meet the coding rule criteria. In operation 380, the one or more processors transmit disease data for the patient to the requesting client device.

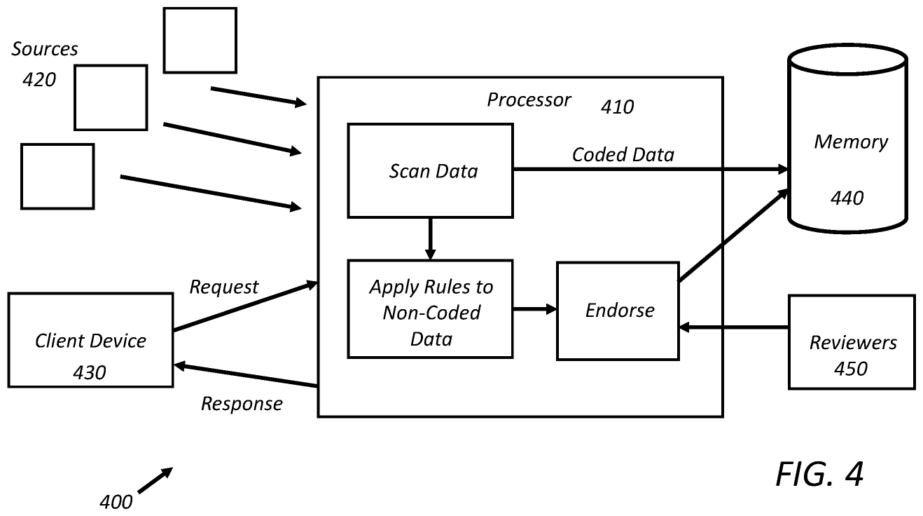

FIG. 4 illustrates a disease discovery system 400 including a processor 410 that receives patient data from sources 420 and patient requests from a client device 430. The processor 410 enables the discovery of diseases that have not been coded as part of the patient record. Even though patient characteristics received by the processor 410 from the sources 420 may not be coded, the processor 410 applies Medicare HCC (Hierarchal Condition Category) logic to the characteristics, provides support for compliance with CQMs (Clinical Quality Measures), and compares the performance of providers with regard to clinical quality and resource use.

The discovery of diseases based on patient characteristics by the processor 410 includes building a longitudinal patient record that is stored in a memory 440, creating value sets, forming a clinical rules engine, applying the clinical rules to the longitudinal patient record, applying post-processing to rules, and finding ICD diagnosis codes for non-coded patient characteristics. Finding and applying correct ICD diagnosis codes by the processor 410 overcomes several technical challenges.

Processor 410 reviews patient data received from the sources 420 and stores coded data in the memory 440 as part of the longitudinal patient record. The processor 410 applies rules to non-coded data and endorsements from reviewers 450 to appropriately assign ICD diagnosis codes to the non-coded data. Once assigned codes, the information can be stored in the memory 440 as part of the longitudinal patient record.

While technical solutions improve operation of the cognitive processing software, these technical solutions also offer clinical decision support to physicians real-time to improve their ability to discover disease, improve their ability to code disease, and improve their ability to offer medical care to patients that meet the denominator for a CQM that have not met the numerator.

It is to be understood that any examples used herein are simply for purposes of explanation and are not intended to be limiting in any way. It is also to be understood that any examples used herein are simply for purposes of explanation and are not intended to be limiting in any way.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, unless otherwise noted, the use of the words “approximate,” “about,” “around,” “substantially,” etc., mean plus or minus ten percent.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A computer-based method for discovering diseases associated with a patient, the method comprising:
receiving, by one or more processors and from a client device, a request for a health assessment of a patient;
aggregating, by one or more processors and from one or more sources, patient data associated with the patient;
scanning, by the one or more processors, the patient data to generate patient disease data for the patient data that includes a list of characteristics identifying a disease that has not been coded in the patient data, the scanning based on a database of parser rules corresponding to interoperability standards and inspection rules corresponding to disease symptoms; and
transmitting, by the one or more processors, the patient disease data to the client device to cause the patient disease data to be presented via the client device.

2. The method of claim 1, wherein the scanning further comprises:
normalizing, by the one or more processors and based on the parser rules, a first set of data extracted from the patient data and a second set of data extracted from the patient data to generate normalized data; and
identifying, by the one or more processors and based on the inspection rules, a disease from the normalized data.

3. The method of claim 1, wherein the patient disease data includes a second list of characteristics identifying a second disease that has not been coded in the patient data.

4. The method of claim 1, wherein each characteristic of the list of characteristics are caused by the disease and/or a response of the patient to the disease.

5. The method of claim 2, further comprising calculating, by the one or more processors, a risk score indicative of a probability for the patient to have a disease, wherein the risk score is calculated by aggregating the patient disease data with demographic characteristics.

6. The method of claim 1, further comprising creating a disease discovery rules engine by converting disease characteristics to a set of rules, rating each rule based on relevance to a specific disease, and receiving endorsements for rules not exclusive to a specific disease based on whether they persist or whether they may be cured.

7. The method of claim 6, further comprising applying a patient record to the disease discovery rules engine.

8. The method of claim 6, further comprising excluding diseases using a post-processing logic rules engine where diseases meet a definition of a particular disease based on expert medical knowledge but do not meet the coding rule criteria.

9. A non-transitory computer readable media with computer-executable instructions embodied thereon, wherein the instructions when executed by a processor of a system cause the system to perform a process comprising:
receiving, from a client device, a request for a health assessment of a patient;
aggregating, from one or more sources, patient data associated with the patient;
scanning the patient data to generate patient disease data for the patient data that includes a list of characteristics identifying a disease that has not been coded in the patient data, the scanning based on a database of parser rules corresponding to interoperability standards and inspection rules corresponding to disease symptoms; and
transmitting the patient disease data to the client device to cause the patient disease data to be presented via the client device.

10. A system comprising:
one or more processors performing a process including:
receiving, from a client device, a request for a health assessment of a patient;
aggregating, from one or more sources, patient data associated with the patient;
scanning the patient data to generate patient disease data for the patient data that includes a list of characteristics identifying a disease that has not been coded in the patient data, the scanning based on a database of parser rules corresponding to interoperability standards and inspection rules corresponding to disease symptoms; and
transmitting the patient disease data to the client device to cause the patient disease data to be presented via the client device.
